(19) United States
(12) Patent Application Publication
(10) Pub. No.: US 2008/0045911 A1
(43) Pub. Date: Feb. 21, 2008

(54) PUNCTAL PLUGS FOR THE DELIVERY OF ACTIVE AGENTS

(76) Inventors: Maureen J. Borgia, Somerville, NJ (US); Hassan Chaouk, Jacksonville, FL (US); Han Cui, Basking Ridge, NJ (US); Walter Laredo, Fort Worth, TX (US); Zhigang Li, Hillsborough, NJ (US); Aruna Nathan, Bridgewater, NJ (US); Michael J. Trezza II, Great Meadows, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/759,281

(22) Filed: Jun. 7, 2007

Related U.S. Application Data
(60) Provisional application No. 60/805,380, filed on Jun. 21, 2006.

(51) Int. Cl. A61F 9/00

(52) U.S. Cl. 604/294

(57) ABSTRACT

The invention provides punctal plugs for the delivery of active agents. The plugs have a body throughout which at least one active agent is dispersed or that is coated with a polymeric material containing at least one active agent.

### RELATED APPLICATION

This application claims priority from provisional application U.S. Ser. No. 60/805,380 filed on Jun. 21, 2006.

### FIELD OF THE INVENTION

The present invention relates to devices suitable for delivering substances to one or more of the eye, nose and throat. In particular, the invention relates to punctal plugs for delivery of at least one active agent.

### BACKGROUND OF THE INVENTION

Human tears are secreted by the lacrimal gland and flow across the surface of the eye to a shallow pool, known as the lacrimal lake, located where the eyelids come together at their inner ends. From there, the tears drain through small openings in each of the eyelids, termed the superior lacrimal punctum and the inferior lacrimal punctum. From the superior and inferior puncta, the tears pass into the superior and inferior lacrimal canaliculus, respectively, which are duct-like pathways that lead to the lacrimal sac. The lacrimal sac is the superior, expanded portion of the nasolacrimal duct, which drains tears into the nasal system. Active agents can thus be delivered to the nose and throat through the lacrimal canaliculi, which lead into the nasolacrimal duct.

Active agents frequently are administered to the eye for the treatment of ocular diseases and disorders. Conventional means for delivering active agents to the eye involve topical application to the surface of the eye. The eye is uniquely suited to topical administration because, when properly constituted, topically applied active agents can penetrate through the cornea and rise to therapeutic concentration levels inside the eye. Active agents for ocular diseases and disorders may be administered orally or by injection, but such administration routes are disadvantageous in that, in oral administration, the active agent may reach the eye in too low a concentration to have the desired pharmacological effect and their use is complicated by significant, systemic side effects, while injections pose the risk of infection.

The majority of ocular active agents are currently delivered topically using eye drops which, though effective for some applications, are inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

### BRIEF DESCRIPTION OF THE DRAWINGS

FIG. 1 is a sectional view of a punctal plug having a body 10. The active agent 18 is dispersed throughout the body.

FIG. 2 is a sectional view of a punctal plug having a body 20 with an enlarged segment 22. The active agent 28 is dispersed throughout the body.

FIG. 3 is a sectional view of a punctal plug having a body 30 with an enlarged segment 32 and a collarette 34. The active agent 38 is dispersed throughout the body 30.

FIG. 4 is a sectional view of a punctal plug having a body 40. The active agent 48 is dispersed throughout the body 40, and all but one portion of the body 41 is coated with a membrane 43.

FIG. 5 is a sectional view of a punctal plug having a body 50 with an enlarged segment 52. The active agent 58 is dispersed throughout the body 50, and all but one portion of the body 51 is coated with a membrane 53.

FIG. 6 is a sectional view of a punctal plug having a body 60 with an enlarged segment 62 and a collarette 64. The active agent 68 is dispersed throughout the body 60, and all but one portion of the body 61 is coated with a membrane 63.

FIG. 7 is a sectional view of a punctal plug having a body 70 with an enlarged segment 72. The body 70 is coated with a polymeric material 78 containing the active agent.

FIG. 8 is a sectional view of a punctal plug having a body 80 with an enlarged segment 82 and a collarette 84. The body and collarette are coated with a polymeric material 88 containing the active agent.

FIG. 9 is a sectional view of a punctal plug having a body 90 with an enlarged segment 92 and a collarette 94. The body contains recesses 95 that increase its surface area. The body and collarette are coated with a polymeric material 98 containing the active agent.

FIG. 10 is a three-dimensional view of the punctal plug depicted two-dimensionally in FIG. 6. The punctal plug has a body 100 with an enlarged segment 102 and a collarette 104. The active agent is dispersed throughout the body 100, and all but one portion of the body 101 is coated with a membrane 103.

### DETAILED DESCRIPTION OF THE INVENTION AND ILLUSTRATIVE EMBODIMENTS

The present invention provides punctal plugs that can be used to deliver active agents to one or both of the nasolacrimal duct and to the tear fluid of the eye. In one embodiment, the invention provides punctal plugs comprising, consisting essentially of, and consisting of: a body having a first end, a second end, and a lateral surface extending between the two ends, wherein at least one active agent is contained throughout the body.

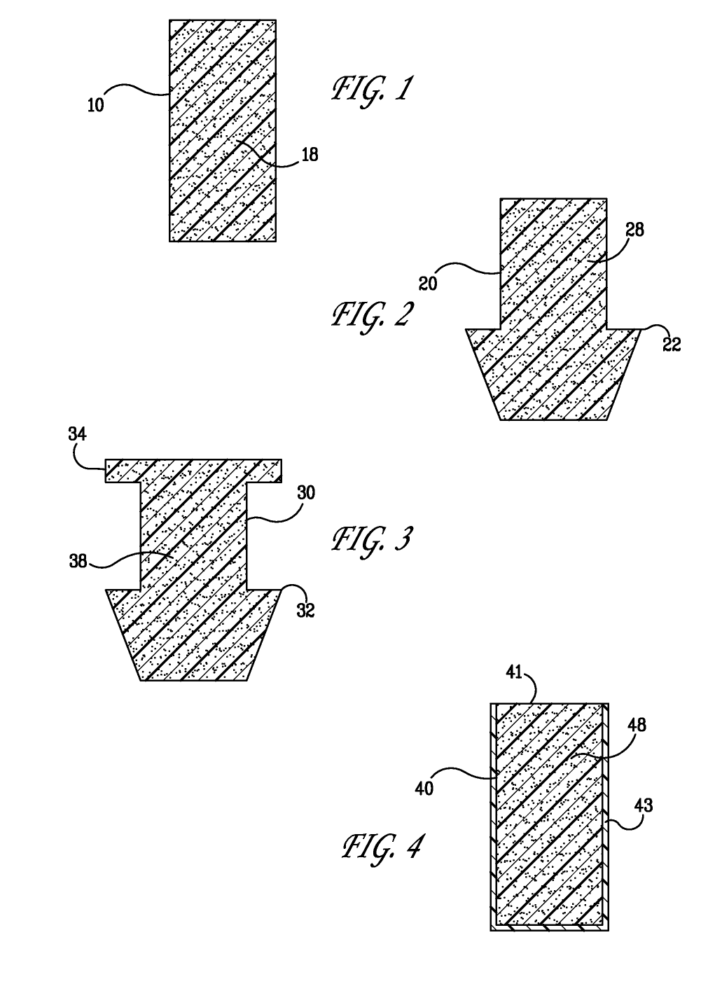

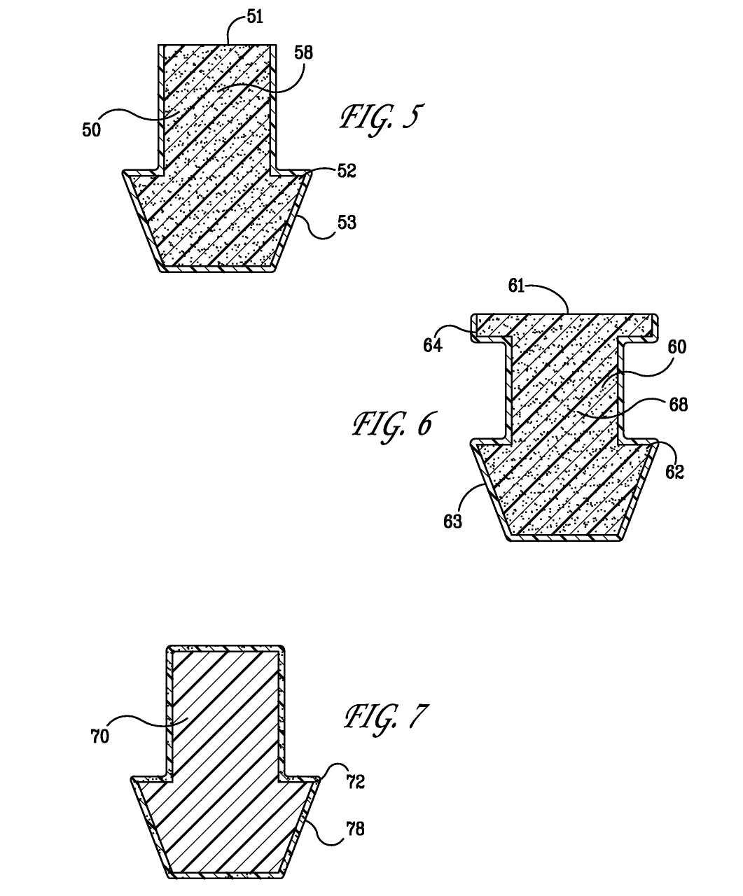

In certain of the punctal plugs and as depicted in FIGS. 1, 2 and 3, the active agent is dispersed throughout the body of the plug, and the active agent is released, for example, when the body dissolves or degrades, or the active agent diffuses from the body, depending upon the material from which the body is made. Alternatively, and as exemplified in FIGS. 4, 5, 6 and 10, the active agent is dispersed throughout the body of the plug and the surface of all but at least one portion of the punctal plug is coated with a membrane that is impermeable to the active agent. The active agent is released through the uncoated portion or portions of the body.

Still further punctal plugs are coated with a polymeric material that comprises the active agent. In one such embodiment and with reference to FIG. 9, the punctal plug has one or more and preferably at least two recesses 95 in the body 90 that increase its surface area. The active agent is released from the coating 98 when the coating dissolves or degrades, or the active agent diffuses from the coating, depending upon the polymeric material from which the coating is made.

For delivery of active agent into the tear fluid of the eye, a punctal plug is inserted into the lacrimal canaliculus and the active agent is released into the tear fluid of the eye. Referring to FIG. 2, for delivery into the tear fluid a collarette 34 is preferably connected to the body of the punctal plugs, and when the punctal plug is inserted into the lacrimal canaliculus. The collarette 34 rests on the exterior of the lacrimal punctum. For delivery of active agent into the nasolacrimal duct, a punctal plug is inserted, preferably deeply, into the lacrimal canaliculus and the active agent is released into the nasolacrimal duct. In particular embodiments of the invention, and as illustrated in FIGS. 2, 3, 5, 6, 7, 8, and 9 for example, the body contains an enlarged segment 22, 32, 52, 62, 72, 82, and 92, respectively, that secures the punctal plug in the lacrimal canaliculus.

As used herein, the term “punctal plug” refers to a device of a size and shape suitable for insertion into the inferior or superior lacrimal canaliculus of the eye through the inferior or superior lacrimal punctum.

As used herein, the term “active agent” refers to an agent capable of treating, inhibiting, or preventing a disorder or a disease. Exemplary active agents include, without limitation, pharmaceuticals and nutraceuticals. Preferred active agents are capable of treating, inhibiting, or preventing a disorder or a disease of one or more of the eye, nose and throat.

As used herein, the phrase “a material that is at least partially water-soluble” refers to a material that exhibits a level of solubility in water sufficient to result in detectable dissolution of the material upon exposure to an aqueous environment.

As used herein, the phrase “a material that is biodegradable” refers to a material that degrades to a detectable degree upon exposure to biologically active substances typically present in mammals.

As used herein, the phrase “a material that is insoluble in water” refers to a material that does not dissolve to a substantial degree upon exposure to water.

As used herein, the phrase “a material that is non-biodegradable” refers to a material that does not degrade to a substantial degree upon exposure to biologically active substances typically present in mammals.

As used herein, the phrase “membrane that is impermeable to active agent” refers to a membrane through which only an insubstantial amount of active agent can pass.

As used herein, the phrase “membrane that is permeable to water” refers to a membrane through which a detectable level of water can pass.

As used herein, the terms “recess,” “recesses,” and all variations thereof, refer to indentations of any size or shape in the body of a punctal plug that effectively increase the surface area of the body.

The present invention encompasses numerous punctal plugs for the delivery of active agent to the tear fluid of the eye or to the nasolacrimal duct. The punctal plugs preferably are inserted into the inferior lacrimal canaliculus, the superior lacrimal canaliculus, or both the inferior and superior lacrimal canaliculi. If the punctal plugs are being used to deliver active agent to the tear fluid of the eye, the punctal plugs preferably have a collarette at one end of the body. The collarette is a portion of the punctal plug that extends radially outwardly from one end of the body to a degree sufficient so that at least a portion of the collarette will extend beyond and be exterior to the lacrimal punctum after insertion of the punctal plug into the lacrimal canaliculus. The portion of the punctal plugs without the collarette is inserted into one of the inferior lacrimal punctum or the superior lacrimal punctum, which are the openings of the lacrimal canaliculus on the margin of each eyelid. Referring to FIG. 3, enlarged segment 32 and body 30 are inserted into one of the punctum, and collarette 34 rests against the exterior of the lacrimal punctum and keeps the punctal plug from slipping down into the lacrimal canaliculus, so that contact between the punctal plug and the tear fluid of the eye is maintained. The collarette can be of any size and shape sufficient to at least partially secure the punctal plug in the lacrimal punctum.

If the punctal plugs are being used to deliver active agent to the nasolacrimal duct, the punctal plugs preferably do not have a collarette so that they may be inserted at a sufficient depth within one or both of the lacrimal canaliculi such that the active agent is released into the lacrimal sac. In FIGS. 1, 2 and 4 are depicted examples of punctal plugs useful for delivery of an active agent to the nasal lacrimal duct.

The numerous punctal plugs of the invention each have various features and advantages. For example, certain punctal plugs have a body with a first end, a second end, and a lateral surface extending between the two ends. The lateral surface preferably has an outer diameter that is substantially circular in shape. A portion of the lateral surface of certain punctal plugs has an outer diameter that is greater than the outer diameter of the remainder of the lateral surface. With reference to FIG. 5, the enlarged portion 52 of the lateral surface anchors or secures the punctal plugs in the lacrimal canaliculus. The enlarged portion can be any size or shape, and can be present on any part of the lateral surface, so long as the enlarged portion at least partially anchors the punctal plug in the lacrimal canaliculus. Conveniently, the enlarged portion may take the shape of an inverted triangle having a flattened apex.

In certain punctal plugs, at least one active agent is disposed within, dispersed throughout, or otherwise contained throughout substantially the whole of the body of the punctal plug, such that the body itself serves as a carrier for the active agent. In certain embodiments of the invention, preferably those in which the punctal plugs have a collarette, the active agent is released from the body into the tear fluid of the eye. The active agent can also be released from the body into the nasolacrimal duct. In particular aspects of the invention, the active agent is released from the body into both the tear fluid of the eye and the nasolacrimal duct.

Depending upon the material from which the body is made, the active agent can be released from the body almost immediately, or the active agent can be released in a sustained manner over a desired period of time. For example, the body can be made of a polymeric material that is at least partially soluble in water. When such a body is exposed to the aqueous environment of the lacrimal canaliculus or the tear fluid, it preferably will dissolve and release the active agent as it dissolves. The solubility in water of the polymeric material from which the body is made typically will be directly proportional to its rate of dissolution. Suitable polymeric materials that are at least partially soluble in water include, without limitation: poly(ethylene glycol); poly(ethylene oxide); poly(propylene gycol); poly(vinyl alcohol); poly(hydroxyethyl methacrylate); poly(vinylpyrrolidone); polyacrylic acid; poly(ethyloxazoline); poly(dimethyl acrylamide); phosolipids including, without limitation, phosphoryl choline derivatives; polysulfobetains; polysaccharides and carbohydrates, such as, for example, hyaluronic acid, dextran, hydroxyethyl cellulose, hydroxyl propyl cellulose, gellan gum, guar gum, heparan sulfate, chondritin sulfate, heparin, and alginate; proteins including, without limitation, gelatin, collagen, albumin, and ovalbumin; and polyamino acids. The polymeric materials in this list typically can be copolymerized or blended with one or more of hydrophobic polymers and monomers.

Alternatively, for those punctal plugs in which the body serves as the carrier for the active agent, the body of the punctal plugs can be made of a biodegradable polymeric material that chemically degrades upon exposure to, for example, biologically active substances typically present in mammals. The biodegradable polymeric materials are preferably hydrolyzable under in vivo conditions. Biodegradation typically occurs more slowly than dissolution, and the body of the plug can be made of biodegradable polymeric materials if slower, more sustained release of the active agent is desired. Suitable biodegradable polymeric materials include, without limitation, polymers and oligomers of glycolide, lactide, epsilon-caprolactone, and other hydroxy acids, and other biologically degradable polymers that yield materials that are non-toxic or present as normal metabolites in the body. Preferred poly(alpha-hydroxy acids) are poly(glycolic acid), poly(2-dioxanone), poly(DL-lactic acid) and poly(L-lactic acid). Other useful materials include poly(amino acids), polycarbonates, poly(anhydrides), poly(orthoesters), poly(phosphazines) and poly(phosphoesters). Polylactones such as poly(epsilon-caprolactone), poly(delta-caprolactone), poly(delta-valerolactone) and poly(gamma-butyrolactone), for example, are also useful, as are chitosan, alginates, collagen, and gelatin. In particular aspects of the invention, the polymeric material of which the body is made can be a mixture of one or more dissolvable and bio-degradable polymers.

The body of those punctal plugs that serves as the carrier for the active agent can alternatively are made of a polymeric material that is insoluble in water and non-biodegradable, but from which the active agent can diffuse. Suitable polymeric materials of this type typically include, without limitation, cross-liked polymers, such as, for example, cross-linked poly(ethylene glycol), poly(ethylene oxide), poly(propylene gycol), poly(vinyl alcohol), poly(hydroxyethyl methacrylate), poly(vinylpyrrolidone), polyacrylic acid, poly(ethyloxazoline), and poly(dimethyl acrylamide). These polymers can be copolymerized or blended with one or both of hydrophobic polymers and monomers. Additional polymeric materials that are insoluble in water and non-biodegradable include, without limitation, silicone; silicone blends; silicone co-polymers, such as, for example, hydrophilic monomers of pHEMA (poly hydroxyethlymethacrylate), polyethylene glycol, polyvinylpyrrolidone, and glycerol; silicone hydrogel polymers such as, for example, those described in U.S. Pat. Nos. 5,962,548, 6,020,445, 6,099,852, 6,367,929, and 6,822,016, incorporated herein in their entireties by reference; phosolipids including, without limitation, phosphoryl choline derivatives; polysulfobetains; polysaccharides and carbohydrates, such as, for example, hyaluronic acid, dextran, hydroxyethyl cellulose, hydroxyl propyl cellulose, gellan gum, guar gum, heparan sulfate, chondritin sulfate, heparin, and alginate; proteins such as, for example, gelatin, collagen, albumin, and ovalbumin; polyamino acids; fluorinated polymers, such as, for example, polytetrafluoroethylene (“PTFE”), polyvinylidene fluoride (“PVDF”), and teflon; polypropylene; polyethylene; nylon; and ethylene vinyl alcohol (“EVA”). Additional examples of suitable polymers that are either or both insoluble in water and non-biodegradable include, without limitation, silicones, polyurethanes, cyanoacrylates, polyacrylic acid, fibrin, and cross-linked proteins, such as, for example, albumin and collagen-gellatin.

The amount of active agent used in the plugs of the invention will depend upon the active agent or agents selected, the desired doses to be delivered via the punctual plug, the desired release rate, and the melting points and solubilities of the active agent and polymeric material. Preferably, the amount used is a therapeutically effective amount meaning an amount effect to achieve the desired treatment, inhibitory, or prevention effect. Typically, amounts of about 0.05 to about 20,000 micrograms of active agents may be used. The ratio of active agent to polymeric material will be about 10 to about 90% and preferably about 20 to about 50%.

Punctal plugs in which the body serves as the carrier for at least one active agent can be manufactured using processes that include techniques such as, for example, solution casting, extrusion, chemical cross-linking through the formation of covalent bonds or ionic bonds, lathing, compression molding, injection molding, liquid injection molding, blow molding, and polymerization, including photo polymerization, thermal polymerization, and ionic- and redox-initiated polymerization. The active agent can be incorporated into the punctal plugs by adding it to the materials that form the body during manufacture of the body, or the active agent can be added to the body of the punctal plugs following their manufacture by, for example, soaking a solution of the active agent into the pre-formed body.

The surface of all but one portion of the body of punctal plugs in which the body serves as the carrier for the active agent can be coated with a membrane that is impermeable to the active agent, so that the active agent is only released from the uncoated portion of the body. The uncoated portion can be any place on the body, including the collarette, if present, depending upon the desired location of release of the active agent. In certain embodiments of the invention, when the punctal plugs are inserted into the lacrimal canaliculus, an uncoated portion of the body faces the eye, and the active agent is released into the tear fluid of the eye. In other aspects of the invention, when the punctal plugs are inserted into the lacrimal canaliculus, an uncoated portion of the body faces the nasolacrimal duct, and the active agent is released into the nasolacrimal duct. In still further embodiments of the invention, when the punctal plugs are inserted into the lacrimal canaliculus, uncoated portions of the body face both the eye and the nasolacrimal duct, and the active agent is released into both the tear fluid of the eye and the nasolacrimal duct.

The coating is preferably made of materials that are impermeable to the active agent including, without limitation, ethylene vinyl alcohol, ethylene-vinylacteate, cellulose derivatives, such as cellulose actetate, polydimethylesiloxane derivatives, and polyurethanes. In certain punctal plugs, the membrane is impermeable to water, and the active agent passively diffuses through the uncoated portion of the body. Such membranes can be made from the materials listed in the previous paragraph.

In other punctal plugs, the membrane is permeable to water but impermeable to the active agent. Following insertion into the lacrimal canaliculus, water diffuses through the membrane into the body to create an osmotic gradient, as described, for example, in U.S. Pat. Nos. 6,923,800 and 5,817,335, incorporated herein by reference in their entireties. The active agent is then forced by the osmotic gradient through the uncoated portion of the body. Membranes that are permeable to water but impermeable to the active agent can be made from materials including, for example, HYTREL® polybutylene terephthalate elastomer, cellulose ethers, cellulose esters, water flux-enhancing polyvinyl acetate copolymers, and ethylene vinyl alcohol.

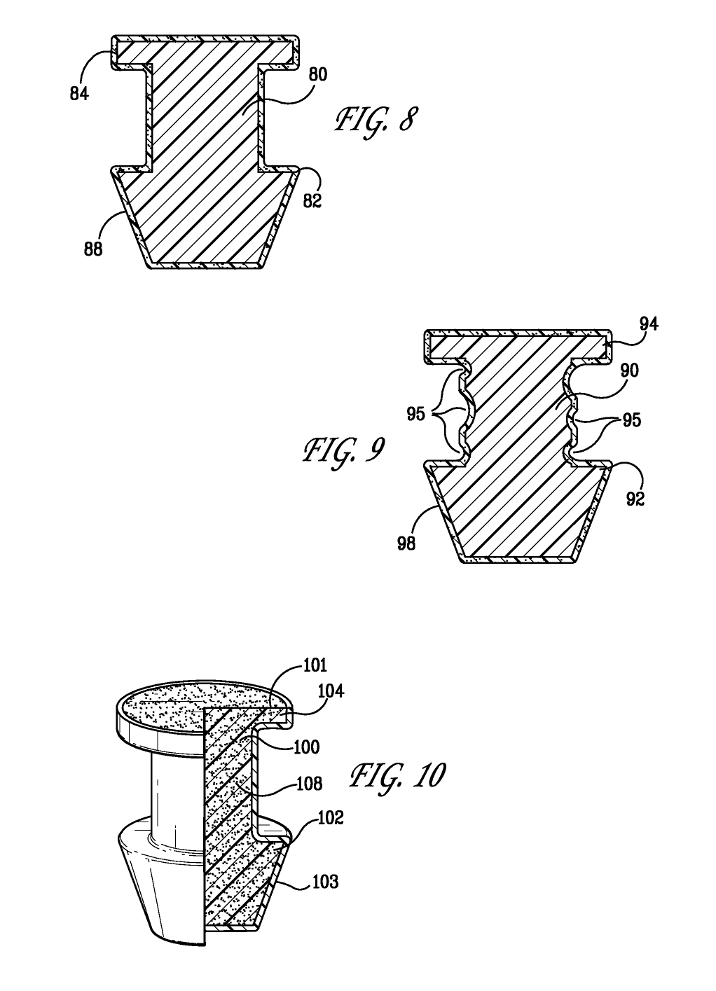

With reference to FIGS. 7 and 8, in certain embodiments the active agent is not contained within the body, but rather the body 70 and 80, respectively, is coated with a polymeric material 78 and 88, respectively, that contains at least one active agent and the body is impermeable to the active agent. The thickness of the coating can be increased, and the size of the body can be proportionately decreased, to increase the amount of active agent that the punctal plug can hold. The coating can thus be of any thickness sufficient to hold a desired amount of active agent. Certain of such punctal plugs can also have recesses in the body that increase its surface area. The recesses can be of any size or shape.

The coating can be made of a polymeric material that is at least partially soluble in water. When such a coating is exposed to the aqueous environment of the lacrimal canaliculus or the tear fluid, it preferably will dissolve and release the active agent as it dissolves. The solubility in water of the polymeric material from which the coating is made typically will be directly proportional to its rate of dissolution. Suitable polymeric materials that are at least partially soluble in water include, without limitation, poly(ethylene glycol); poly(ethylene oxide); poly(propylene gycol); poly(vinyl alcohol); poly(hydroxyethyl methacrylate); poly(vinylpyrrolidone); polyacrylic acid; poly(ethyloxazoline); poly(dimethyl acrylamide); phosolipids including, without limitation, phosphoryl choline derivatives; polysulfobetains; polysaccharides and carbohydrates including, without limitation, hyaluronic acid, dextran, hydroxyethyl cellulose, hydroxyl propyl cellulose, gellan gum, guar gum, heparan sulfate, chondritin sulfate, heparin, and alginate; proteins including, without limitation, gelatin, collagen, albumin, and ovalbumin; and polyamino acids. The polymeric materials in this list can typically be copolymerized or blended with one or both of hydrophobic polymers and monomers.

Alternatively, the coating can be made of a biodegradable polymeric material that chemically degrades upon exposure to, for example, biologically active substances typically present in mammals. The biodegradable polymeric materials are preferably hydrolyzable under in vivo conditions. Biodegradation typically occurs more slowly than dissolution, and the coating can thus be made of biodegradable materials if slower, more sustained release of the active agent is desired. Suitable biodegradable polymeric materials include, without limitation, polymers and oligomers of glycolide, lactide, epsilon-caprolactone, and other hydroxy acids, and other biologically degradable polymers that yield materials that are non-toxic or present as normal metabolites in the body. Preferred poly(alpha-hydroxy acids) are poly(glycolic acid), poly(2-dioxanone); poly(DL-lactic acid) and poly(L-lactic acid). Other useful materials include poly(amino acids), polycarbonates, poly(anhydrides), poly(orthoesters), poly(phosphazines) and poly(phosphoesters). Polylactones such as poly(epsilon-caprolactone), poly(delta-caprolactone), poly(delta-valerolactone) and poly(gamma-butyrolactone), for example, are also useful, as are chitosan, alginates, collagen, and gelatin. In particular aspects of the invention, the polymeric material of which the coating is comprised can comprise a mixture of one or more dissolvable and bio-degradable polymers.

The coating can alternatively be made of a polymeric material that is insoluble in water and non-biodegradable, but from which the active agent can diffuse. Suitable polymeric materials of this type typically include, without limitation, cross-liked polymers including, without limitation, cross-linked poly(ethylene glycol), poly(ethylene oxide), poly(propylene gycol), poly(vinyl alcohol), poly(hydroxyethyl methacrylate), poly(vinylpyrrolidone), polyacrylic acid, poly(ethyloxazoline), and poly(dimethyl acrylamide). These polymers can be copolymerized or blended with one or both of hydrophobic polymers and monomers. Additional polymeric materials that are insoluble in water and non-biodegradable include, without limitation, silicone; silicone blends; silicone co-polymers, such as, for example, hydrophilic monomers of pHEMA (poly hydroxyethlymethacrylate), polyethylene glycol, polyvinylpyrrolidone, and glycerol; silicone hydrogel polymers including, without limitation, those described in U.S. Pat. Nos. 5,962,548, 6,020,445, 6,099,852, 6,367,929, and 6,822,016, incorporated herein in their entireties by reference; phosolipids, including, without limitation, phosphoryl choline derivatives; polysulfobetains; polysaccharides and carbohydrates, such as, for example, hyaluronic acid, dextran, hydroxyethyl cellulose, hydroxyl propyl cellulose, gellan gum, guar gum, heparan sulfate, chondritin sulfate, heparin, and alginate; proteins including, without limitation, gelatin, collagen, albumin, and ovalbumin; polyamino acids; fluorinated polymers, such as, for example, PTFE, PVDF, and teflon; polypropylene; polyethylene; nylon; and EVA. Additional examples of suitable polymers that are insoluble in one or both of water and non-biodegradable includw, without limitation, silicones, polyurethanes, cyanoacrylates, polyacrylic acid, fibrin, and cross-linked proteins, such as, for example, albumin and collagen-gellatin.

For punctal plugs in which the body is coated with a polymeric material that is made of at least one active agent, the body is first manufactured using suitable processes that can include, for example, solution casting, extrusion, chemical cross-linking through the formation of covalent bonds or ionic bonds, lathing, compression molding, injection molding, liquid injection molding, blow molding, and polymerization, including photo polymerization, thermal polymerization, and ionic- and redox-initiated polymerization. The body is then coated with a polymeric material that contains the active agent using any of a variety of process including, without limitation, dip coating, spin coating, vapor deposition coating, adsorption, incorporation of laminates, adhesion of a preformed coating, plasma coating, powder coating, and spray coating, including electro-spraying. The coating can be polymerized onto the surface of the punctal plug, adsorbed via mechanical interlock, precipitated via the evaporation of a solvent carrier, precipitated or solidified on cooling, chemically bonded via the use of cross-linking agents, or bound via an adhesive. Alternatively, the coating can be in the form of a non-bonded sleeve around one or both of the body and collarette of the plug.

The amount of active agent used in the plugs of the invention will depend upon the active agent or agents selected, the desired doses to be delivered via the punctual plug, the desired release rate, and the melting points of the active agent and material used to form the plug. Preferably, the amount used is a therapeutically effective amount meaning an amount effective to achieve the desired treatment, inhibitory, or prevention effect. Typically, amounts of about 0.05 to about 8,000 micrograms of active agents may be used.

The punctal plugs described herein can be used to deliver various active agents for the one or more of the treatment, inhibition, and prevention of numerous diseases and disorders. Each punctal plug can be used to deliver at least one active agent and can be used to deliver different types of active agents. For example, the punctal plugs can be used to deliver azelastine HCl, emadastine difumerate, epinastine HCl, ketotifen fumerate, levocabastine HCl, olopatadine HCl, pheniramine maleate, and antazoline phosphate for one or more of the treatment, inhibition, and prevention of allergies. The punctal plugs can be used to deliver mast cell stabilizers, such as, for example, cromolyn sodium, lodoxamide tromethamine, nedocromil sodium, and permirolast potassium.

The punctal plugs can be used to deliver mydriatics and cycloplegics, such as, for example, henylephrine, atropine sulfate, homatropine, scopolamine HBr, cyclopentolate HCl, tropicamide, and phenylephrine HCl. The punctal plugs can be used to deliver ophthalmic dyes such as, for example and without limitation, rose begal, sissamine green, indocyanine green, fluorexon, and fluorescein.

The punctal plugs can be used to deliver corticosteroids such as, for example, dexamethasone sodium phosphate, dexamethasone, fluoromethalone, fluoromethalone acetate, loteprednol etabonate, prednisolone acetate, prednisolone sodium phosphate, medrysone, rimexolone, and fluocinolone acetonide. The punctal plugs can be used to deliver non-steroidal anti-inflammatory agents such as, for example and without limitation, flurbiprofen sodium, suprofen, diclofenac sodium, ketorolac tromethamine, cyclosporine, rapamycin methotrexate, azathioprine, and bromocriptine.

The punctal plugs can be used to deliver anti-infective agents such as, for example and without limitation, tobramycin, moxifloxacin, ofloxacin, gatifloxacin, ciprofloxacin, gentamicin, sulfisoxazolone diolamine, sodium sulfacetamide, vancomycin, polymyxin B, amikacin, norfloxacin, levofloxacin, sulfisoxazole diolamine, sodium sulfacetamide tetracycline, doxycycline, dicloxacillin, cephalexin, amoxicillin/clavulante, ceftriaxone, cefixime, erythromycin, ofloxacin, azithromycin, gentamycin, sulfadiazine, and pyrimethamine.

The punctal plugs can be used to deliver agents for the one or more of the treatment, inhibition, and prevention of glaucoma including, without limitation, epinephrines, including, for example: dipivefrin; alpha-2 adrenergic receptors, including, for example, aproclonidine and brimonidine; betablockers, including, for example, betaxolol, carteolol, levobunolol, metipranolol, and timolol; direct miotics, including, for example, carbachol and pilocarpine; cholinesterase inhibitors, including, for example, physostigmine and echothiophate; carbonic anhydrase inhibitors, including, for example, acetazolamide, brinzolamide, dorzolamide, and methazolamide; prostoglandins and prostamides, including, for example, latanoprost, bimatoprost, uravoprost, and unoprostone cidofovir.

The punctal plugs can be used to deliver antiviral agents, including, without limitation, fomivirsen sodium, foscarnet sodium, ganciclovir sodium, valganciclovir HCl, trifluridine, acyclovir, and famciclovir. The punctal plugs can be used to deliver local anesthetics, including, without limitation, tetracaine HCl, proparacaine HCl, proparacaine HCl and fluorescein sodium, benoxinate and fluorescein sodium, and benoxnate and fluorexon disodium. The punctal plugs can be used to deliver antifungal agents, including, for example, fluconazole, flucytosine, amphotericin B, itraconazole, and ketocaonazole.

The punctal plugs can be used to deliver analgesics including, without limitation, acetaminophen and codeine, acetaminophen and hydrocodone, acetaminophen, ketorolac, ibuprofen, and tramadol. The punctal plugs can be used to deliver vasoconstricors including, without limitation, ephedrine hydrochloride, naphazoline hydrochloride, phenylephrine hydrochloride, tetrahydrozoline hydrochloride, and oxymetazoline. Finally, the punctal plugs can be used to deliver vitamins, antioxidants, and nutraceuticals including, without limitation, vitamins A, D, and E, lutein, taurine, glutathione, zeaxanthin, fatty acids and the like.

The active agents delivered by the punctal plugs can be formulated to contain excipients including, without limitation, synthetic and natural polymers, including, for example, polyvinylalcohol, polyethyleneglycol, polyacrylic acid, hydroxymethyl cellulose, glycerine, hypromelos, polyvinylpyrrolidone, carbopol, propyleneglycol, hydroxypropyl guar, glucam-20, hydroxypropyl cellulose, sorbitol, dextrose, polysorbate, mannitol, dextran, modified polysaccharides and gums, phosolipids, and sulphobetains.

What is claimed is:

1. A punctal plug, comprising a body having a first end, a second end, and a lateral surface extending between the two ends, and at least one active agent contained throughout the body.

2. The punctal plug of claim 1, wherein the body is comprised of a polymeric material that is at least partially water-soluble, dissolves over time, and releases the active agent as it dissolves.

3. The punctal plug of claim 1, wherein the body is comprised of a polymeric material that is biodegradable, degrades over time, and releases the active agent as it degrades.

4. The punctal plug of claim 1, wherein the body is comprised of a polymeric material that is insoluble in water and non-biodegradable and the active agent passively diffuses from the body.

5. The punctal plug of claim 1, wherein a portion of the lateral surface has an outer diameter that is greater than that of the remainder of the lateral surface and the enlarged portion of the lateral surface secures the punctal plug in the lacrimal canaliculus when the punctal plug is inserted in the lacrimal canaliculus.

6. The punctal plug of claim 1, wherein the active agent is released into the tear fluid of the eye.

7. The punctal plug of claim 1, wherein the active agent is released into the nasolacrimal duct.

8. The punctal plug of claim 1, wherein the active agent is released into both the tear fluid of the eye and the nasolacrimal duct.

9. The punctal plug of claim 1, further comprising a collarette at the first end of the body.

10. A punctal plug, comprising a body having a first end, a second end, and a lateral surface extending between the two ends, at least one active agent is contained throughout the body, and the surface of all but at least one portion of the body is coated with a membrane that is impermeable to the active agent.

11. The punctal plug of claim 10, wherein the lateral surface has an outer diameter that is substantially circular in shape.

12. The punctal plug of claim 10 or 11, wherein the body is comprised of a polymeric material that is at least partially water-soluble, dissolves over time, and releases the active agent through an uncoated portion of the body as it dissolves.

13. The punctal plug of claim 10 or 1 1, wherein the body is comprised of a polymeric material that is biodegradable, degrades over time, and releases the active agent through an uncoated portion of the body as it degrades.

14. The punctal plug of claim 10 or 11, wherein the body is comprised of a polymeric material that is insoluble in water and non-biodegradable.

15. The punctal plug of claim 14, wherein the active agent passively diffuses through an uncoated portion of the body.

16. The punctal plug of claim 14, wherein the membrane is permeable to water, water diffuses through the membrane into the body to create an osmotic gradient, and the active agent is forced by the osmotic gradient through an uncoated portion of the body.

17. The punctal plug of claim 10 or 11, wherein a portion of the lateral surface has an outer diameter that is greater than that of the remainder of the lateral surface and the enlarged portion of the lateral surface secures the punctal plug in the lacrimal canaliculus when the punctal plug is inserted in the lacrimal canaliculus.

18. The punctal plug of claim 10 or 1 1, wherein when the punctal plug is inserted into the lacrimal canaliculus, an uncoated portion of the body faces the eye, and the active agent is released into the tear fluid of the eye.

19. The punctal plug of claim 10 or 11, wherein when the punctal plug is inserted into the lacrimal canaliculus, an uncoated portion of the body faces the nasolacrimal duct, and the active agent is released into the nasolacrimal duct.

20. The punctal plug of claim 10 or 11, wherein when the punctal plug is inserted into the lacrimal canaliculus, an uncoated portion of the body faces the eye, another uncoated portion of the body faces the nasolacrimal duct, and the active agent is released into both the tear fluid of the eye and the nasolacrimal duct.

21. The punctal plug of claim 10 or 1 1, further comprising a collarette at the first end of the body.

22. A punctal plug, comprising a body having a first end, a second end, and a lateral surface extending between the two ends and a portion of the lateral surface is of a greater dimension that the remainder of the lateral surface and wherein the body is coated with a polymeric material comprising at least one active agent, and the body is impermeable to the active agent.

23. The punctal plug of claim 22, wherein the lateral surface has an outer diameter that is substantially circular in shape.

24. The punctal plug of claim 22 and 22, wherein the coating is comprised of a polymeric material that is at least partially water-soluble, dissolves over time, and releases the active agent as it dissolves.

25. The punctal plug of claim 22 and 23, wherein the coating is comprised of a polymeric material that is biodegradable, degrades over time, and releases the active agent as it degrades.

26. The punctal plug of claim 22 and 23, wherein the coating is comprised of a polymeric material that is insoluble in water and non-biodegradable and the active agent passively diffuses from the coating.

27. The punctal plug of claim 22 and 23, wherein the portion of the lateral surface that has an outer diameter greater than that of the remainder of the lateral surface secures the punctal plug in the lacrimal canaliculus when the punctal plug is inserted in the lacrimal canaliculus.

28. The punctal plug of claim 22 and 23, wherein the body has one or more recesses that increase its surface area.

29. The punctal plug of claim 22 and 23, wherein the active agent is released into the tear fluid of the eye.

30. The punctal plug of claim 22 and 23, wherein the active agent is released into the nasolacrimal duct.

31. The punctal plug of claim 22 and 23, wherein the active agent is released into both the tear fluid of the eye and the nasolacrimal duct.

32. The punctal plug of claim 22 and 23, further comprising a collarette at the first end of the body.

33. A method comprising inserting a punctal plug according to claim 1 into the lacrimal canaliculus.

34. A method comprising inserting a punctal plug according to claim 10 or 11 into the lacrimal canaliculus.

35. A method comprising inserting a punctal plug according to claim 22 or 23 into the lacrimal canaliculus.
